(19) United States
(12) Patent Application Publication
(10) Pub. No.: US 2019/0029893 A1
(43) Pub. Date: Jan. 31, 2019

(54) DISPOSABLE SANITARY PAD FOR WOMEN

(71) Applicant: JOAN WARREN, STOCKTON, CA (US)

(72) Inventor: JOAN WARREN, STOCKTON, CA (US)

(21) Appl. No.: 15/662,913

(22) Filed: Jul. 28, 2017

(51) Int. Cl. A61F 13/476; A61F 13/56

(52) U.S. Cl. CPC A61F 13/476; A61F 2013/51407; A61F 13/5616

(57) ABSTRACT

A disposable sanitary pad to removably attach with an undergarment of a user for absorbing the body fluids and cleansing proposes includes a central pad portion having a number absorbent layers and a fluid pervious layer attached to a fluid impervious flexible base using an attachment means. An exterior surface of the fluid impervious flexible base is provided with adhesive strips at a top, bottom, and center portion and at corners to allow the users to attach the disposable sanitary pad by longitudinally covering the crotch area of the undergarment. A pair of flexible wings having adhesives is attached to side portions of the fluid impervious flexible base to maintain a laterally stretched position of the disposable sanitary pad covering the crotch area of the undergarment. A pouch having deodorized wet wipes is removably attached to the central pad portion for handling and disposal of the disposable sanitary pad with hygienic after use.

### (B) CROSS REFERENCE TO RELATED APPLICATIONS

Not applicable

### (C) FEDERALLY SPONSORED RESEARCH AND DEVELOPMENT

Not applicable.

### (D) MICROFICHE

Not applicable

### (E) BACKGROUND OF THE INVENTION

### Field of the Invention

The present invention relates generally to a disposable sanitary pad, more specifically, to a disposable sanitary pad having a number of adhesive points to properly cover and attach over a crotch area of an undergarment of a user for collecting the body fluids and cleansing the subject area.

### Description of the Related Art

Absorbent articles known as, sanitary napkin or a sanitary pad or a menstrual pad or pad, are commonly worn by women of child bearing age and other older and disabled women for conveniently collecting their body fluids and cleansing the subject area. Women wear these absorbent articles to cover the crotch area and are supported using their undergarments to absorb the body fluids. These absorbent articles are designed to absorb and retain liquid and other discharges from the human body and to prevent body and clothing soiling or to prevent the body fluids from coming in contact with the clothing or other body parts of the wearer. Some of the absorbent articles existing in the market enable the women to wear the absorbent articles with their undergarments by properly positioning the absorbent article within the undergarment and to cover the area between the legs i.e. the crotch area of the wearer. However, such absorbent articles placed within the undergarment of the wearer may get dislocated or displaced during physical activities such as walking, running, etc. performed by the wearer. This results in the leakage or spreading of the body fluids into the undergarment or clothing or body parts of the wearer.

Different types of disposable pads exist in the market some of which are provided with a sticky patch on the backside of the pad for attaching or sticking onto the interior of the undergarment of the user. In such disposable pad products, the sticky patch is usually covered using a removable paper, which can be manually removed for sticking the underside of the pad to the undergarment of the user. However, the existing disposable pads are provided with sticky patches at one or two locations, such as at the center, on the backside of the pad. This may results in the easy dislocation or disorientation of the pad from the attached position in the undergarment of the user during normal usage or during movement of the user. This in turn causes rubbing of the pad against the skin of the user, uncomfortable placement of the pad or leakage or spreading of the body fluid to the undergarment of the user. Moreover, such pads are difficult to remove and dispose after use as it may soil the hands of the user while removing the pad from the undergarment of the user.

Another type of sanitary pad existing in the market is provided with flaps that extend laterally from the central absorbent means and are designed to be folded around the edges at the crotch area of the undergarment of the user. In some cases, the flaps are provided with an attachment means such as sticky patches for affixing the flaps to the underside of the undergarment at the crotch area of the user. In some other designs, the flaps are provided with enough length to folder around the undergarment of the user at the crotch area and attach together to prevent the body fluids from soiling the side portions or the edges of the undergarment of the user. However in some instances, some users find it to be difficult to attach the flaps to the underside of the crotch of their panties. This can be due to factors such as the tendency for the adhesive fasteners on the flaps to stick to themselves or to other parts of the sanitary napkin. As a result, some women still prefer a sanitary napkin without flaps having adhesives. In addition, the folding of the flaps around the crotch area of the undergarment of the user may cause shrinkage of the pad at the crotch area during the walking, sitting or any other activity of the user. This in turn affects the fluid absorption capability of the pad and may cause leakage of the body fluids to the undergarments, body parts and the clothing of the user. Therefore, there is a need for a disposable sanitary pad, which provides an alternative to sanitary napkins having conventional side flaps and improperly placed sticky patches at the underside of the pad while still providing the adequate protection from the body fluids reaching the undergarments of the user.

An improved type of sanitary pads disclosed by the prior arts includes flaps commonly for providing better protection against soiling as compared to sanitary pads without flaps and sticky patches at one or more locations of the pad. However some users find that applying sanitary pads having flaps is inconvenient for various reasons. For instance, some women find it to be difficult to attach the flaps to the underside of the crotch of their panties. This can be due to factors such as the tendency for the adhesive fasteners on the flaps to stick to themselves or to other parts of the sanitary napkin. Furthermore, the adhesive fasteners on the flaps also have a tendency to come unglued after an extended use and get stuck on the wearer's skin. As a result, some women still prefer a sanitary napkin without flaps. Some women who generally prefer a sanitary napkin with flaps, occasionally, such as during periods of light flow, prefer a sanitary napkin without flaps. Hence there exists a need for an improved sanitary pad that can be conveniently attached to the undergarments of the user for absorbing the body fluids. The needed sanitary pad would allow the users to properly place the pad within the undergarment of the user and fix the position of the pad using multiple means such as using multiple sticking or attaching points and flexible wings. The needed sanitary pad would maintain the position within the undergarment, covering the crotch area of the user, even after extended periods of usage and during movement or physical activity of the user. Further the needed sanitary pad would solve another problem associated with existing products by maintaining the position and attachment to the undergarment at different conditions such as during wet and dry conditions alike. The below patent literature, which are herein incorporated by reference for their supportive teachings, provides several variations of sanitary pads, attachable to the undergarment of the users, to solve some of these problems.

U.S. Pat. No. 5,650,223 A, titled “Absorbent articles having undergarment covering components with zones of extensibility”, issued to the Procter & Gamble company discloses a sanitary napkin that have undergarment covering components or ‘side wrapping elements’ that automatically wrap the sides of a wearer's panties and provide an alternative to conventional side flaps. The side wrapping elements have at least one zone of extensibility and at a region therein that is stiffer than the zone of extensibility. However the above-disclosed patent failed to disclose a means to position the sanitary napkin within the undergarment during extended periods of usage. Further the application also failed to disclose a means to dispose the sanitary napkin after use without soiling the hands of the user.

Another International patent application with application number WO 2016016903 A2, titled “An improved disposable object”, filed by Atik Zalavadia shows an improved disposable object having two attachments. One attachment is sticked on the release paper of the sticky patch on the bottom side of disposable object whereby this attachment is easily removable from the disposable object allowing the disposable object to stick on the intended material such as panty through sticky patch of the disposable object and keep it sticked in proper position. A second attachment helps the wings to stick properly on under garment by providing thickness and proper grip which placed on the upper side of the disposable object made up of any skin friendly material such as cotton cloth or fiber. An irritation reducing material associated with the disposable object solves the problem of irritation and rashes between the legs and backside at butt part and absorbs sweat and helps to keep the skin dry. However the above patent application failed to disclose a means to attach and retain a position of the sanitary pad within the undergarment of the user.

Yet another U.S. Pat. No. 6,558,364 B1, titled “Sanitary undergarment”, filed by Cathy D. Santa Cruz and Usha Mehta to shows a sanitary undergarment which is usable with an absorbent pad having lateral extension wings. The undergarment having a first and a second crotch portion between which the lateral extension wings can be inserted and held in a secure manner by adhesive. The undergarment can be boxer shorts or a panty. However, the application failed disclose a reliable means for holding the sanitary pad to the undergarment of the user in a longitudinal fashion, covering the crotch area of the user.

Hence, none of the above inventions and patents, taken either singly or in combination, is seen to disclose an invention with an object to provide a disposable sanitary pad having a means for maintaining an attachment position at the crotch area of the undergarment during extended hours of use, during movement and physical activities of the user and during different conditions such as wet and dry conditions alike. Moreover, the needed system would allow the users to detach and dispose the sanitary pad after use without soiling the hands of the user. Hence there exists a need for an improved disposable sanitary pad, which solves the above said and other additional problems associated with the use of the sanitary pad.

### (F) SUMMARY OF THE INVENTION

The present invention relates to a disposable sanitary pad for use with an undergarment of a user to collect the body fluids from the user. The present disposable sanitary pad is a convenient and appropriately designed undergarment protector that keeps women free from embarrassing body function situations. The present disposable sanitary pad is designed to attach with an interior of the undergarment of the user to effectively collect the body fluids and thereby preventing the leakage of the body fluids on to the undergarment of the user. The present disposable sanitary pad allows the users to properly place the disposable sanitary pad and maintain the attached position of the disposable sanitary pad within the undergarment of the user. The present disposable sanitary pad further allows easy and hygienic disposal of the disposable sanitary pad after use. The present disposable sanitary pad enables the users to comfortably place the pad within the undergarment covering a crotch area of the undergarment thereby preventing the shifting of the sanitary pad during movement of the user.

The present disposable sanitary pad configured to removably attach within the undergarment of the user includes a central pad portion having a substantially oblong shape to cover the crotch area of the user. The central pad portion of the disposable sanitary pad includes one or more fluid absorption layers for absorbing the body fluids from the user. The central pad portion of the disposable sanitary pad includes three major layers, such as a fluid impervious flexible base, the absorbent layers and a fluid pervious layer forming a top surface of the central pad portion. The fluid impervious flexible base is provided with a substantially oblong shape sufficient to cover the crotch area of the user. The fluid impervious flexible base has an exterior surface and an interior surface, which supports the top layers including the absorbent layers and the fluid pervious layer for absorbing the body fluids. The edges of the fluid pervious layer and the absorbent layers are attached together using an attachment means, such as, stitching or gluing, to the interior surface of the fluid impervious flexible base. The fluid impervious flexible base is provided with an oblong shape having a top portion, a bottom portion and a pair of side portions to properly cover the crotch area of the undergarment of the user. The top portion and bottom portion at exterior surface of the fluid impervious flexible base is provided with adhesive strips to allow the users to properly attach the disposable sanitary pad to the crotch area of the undergarment. The fluid impervious flexible base of the disposable sanitary pad further includes a pair of flexible wings attached to the pair of side portions of the fluid impervious flexible base. The flexible wings are covered with an adhesive to attach with the undergarment of the user to maintain the position of the disposable sanitary pad covering the crotch area of the undergarment. The fluid impervious flexible base of the disposable sanitary pad is further provided with a pair of elongated adhesive strips longitudinally attached to the exterior surface to hold the central pad portion of the present disposable sanitary pad at the center portion of the crotch area of the undergarment. The present disposable sanitary pad is provided with a pouch removably attached to the exterior surface of the fluid impervious flexible base to store one or more deodorized wet wipes for use during handling and disposal of the disposable sanitary pad.

A number of features of the present invention based on one or more embodiment are discussed below. However, there may be additional objects, which are not listed below and disclosed in the following detailed description and claims section of the application, can form a main feature of the present invention.

The primary object of the present invention is to provide a disposable sanitary pad for removably attaching with an undergarment of a user for effectively collecting the body fluids from the user.

A second feature of the present invention is to provide a disposable sanitary pad having a central pad portion with multiple layers including a fluid impervious flexible base, a number of absorbent layers and a fluid pervious top layer to absorb the body fluids from the user.

Yet another feature of the present invention is to provide a disposable sanitary pad having a number of adhesive strips attached to different location on exterior surface of the fluid impervious flexible base for proper positioning of the pad within the undergarment of the user.

A fourth feature of the present invention is to provide a disposable sanitary pad having a pair of flexible wings capable of being attached to the undergarment of the user for properly covering the crotch area of the undergarment when in use.

A fifth feature of the present invention is to provide a disposable sanitary pad having a means for safely handling and disposing the sanitary pad after use.

Yet another feature of the present invention is to provide a sanitary pad having additional cleaning wipes to use while removing and disposing the sanitary pad with hygiene.

A seventh feature of the present invention is to provide a disposable sanitary pad having a biodegradable pouch to collect the sanitary pad after use for easy disposal.

Another feature of the present invention is to provide a disposable sanitary pad having adhesives provided at a number of locations on the exterior surface of the fluid impervious flexible base coming in contact with the undergarment of the user to properly cover the crotch area of the undergarment to safely and effectively collect the body fluids.

Yet another feature of the present invention is to provide a disposable sanitary pad with flexible wings attachable to the either sides of the crotch area of the undergarment to cover the crotch area and area on either sides of the crotch area towards the pair of legs of the user.

A tenth feature of the present invention is to provide a disposable sanitary pad that is easy and comfortable to wear, easy to remove and dispose after use, and also designed with contoured surface to completely absorb the body fluids when in contact with the body of the user.

Another feature of the present invention is to provide a disposable sanitary pad having attached hygienic measures such as deodorized disinfectant wet wipes to properly handle the disposal of the pad with hygiene.

Yet another feature of the present invention is to provide an improved disposable sanitary pad configured to attach with an undergarment covering the crotch area and is fabricated from skin friendly and fabric friendly materials.

Another feature of the present invention is to provide an improved disposable sanitary pad that provides extra grip with the skin and the undergarments during movements and activities performed by the user.

Another important feature of the present invention is to provide a disposable sanitary pad capable of being attachable to the interior of an undergarment of a user in a longitudinally and transversally stretched condition completely covering the crotch area of the undergarment.

Yet another important feature of the present invention is to provide a disposable sanitary pad, which is fabricated from rash free, allergic free and hygienic materials offering adequate absorption capabilities.

The present disposable sanitary pad is fabricated to removably attach to the undergarment of the user for properly covering the crotch area of the undergarment for complete absorption of the body fluids from the user when in use. A preferred method of using the disposable sanitary pad with the undergarment of the user includes the steps of providing the disposable sanitary pad having the central pad portion with adhesives provided on the exterior surface of the fluid impervious flexible base and the wings on either side of the central pad portion. The users can place the disposable sanitary pad within the undergarment covering the crotch area and the wings directing towards either leg openings after detaching the pouch and associated deodorized wet wipes. The users can remove the adhesive strip coverings on the exterior surface of the fluid impervious flexible base and the wings once the disposable sanitary pad is properly poisoned within the undergarment covering the crotch area. The users can then wear the undergarment with the disposable sanitary pad for a desired period of time and can easily be detached from the undergarment with the help of the deodorized wet wipes provided with the disposable sanitary pad. The disposable sanitary pad can then be put inside the pouch for easy disposal and the deodorized wet wipes allow the proper handling of the pad with hygiene.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

These together with other feature of the invention, along with the various features of novelty, which characterize the invention, are pointed out with particularity in the disclosure. For a better understanding of the invention, its operating advantages and the specific objects attained by its uses, reference should be had to the accompanying drawings and descriptive matter in which there are illustrated preferred embodiments of the invention.

### (G) BRIEF DESCRIPTION OF THE DRAWINGS

To further clarify various aspects of some example embodiments of the present invention, a more particular description of the invention will be rendered by reference to specific embodiments thereof that are illustrated in the appended drawing. It is appreciated that the drawing depicts only illustrated embodiments of the invention and are therefore not to be considered limiting of its scope. Elements in the figures have not necessarily been drawn to scale in order to enhance their clarity and improve understanding of these various elements and embodiments of the invention. Furthermore, elements that are known to be common and well understood to those in the industry are not depicted in order to provide a clear view of the various embodiments of the invention, thus the drawings are generalized in form in the interest of clarity and conciseness. The invention will be described and explained with additional specificity and detail through the use of the accompanying drawing in which:

FIG. 1 illustrates a top view of a disposable sanitary pad, according to a preferred embodiment of the present invention;

FIG. 2 illustrates the disposable sanitary pad attached within the undergarment of the user, according to a preferred embodiment of the present invention; and

FIG. 3 illustrates a flowchart showing a method of comfortably placing the disposable sanitary pad within the undergarment of the user, according to a preferred embodiment of the present invention.

### (H) DETAILED DESCRIPTION OF THE INVENTION

In the following detailed description, reference is made to the accompanying drawings that form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, composition, fabrication method and other changes may be made without departing from the spirit and scope of the present invention. The following detailed description is, therefore, not to be taken in a limiting sense, and the scope of the present invention is defined by the appended claims and their equivalents.

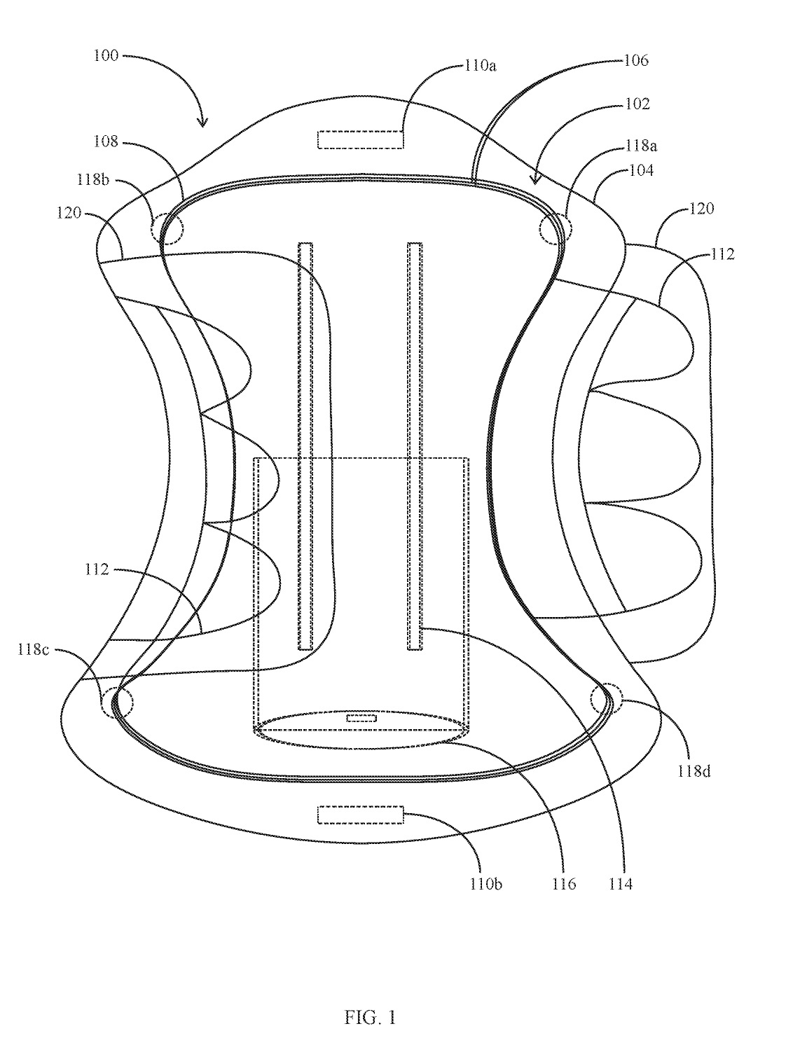

The present invention relates to a disposable sanitary pad for use with an undergarment of a user to collect the body fluids produced by the user. FIG. 1 illustrates a top view of the disposable sanitary pad 100, according to a preferred embodiment of the present invention. The present disposable sanitary pad 100 is a convenient and appropriately designed undergarment protector that keeps women free from embarrassing body function situations, such as, but not limited to, during the menstrual periods. The present disposable sanitary pad 100 is designed to attach with an interior of the undergarment of the user to effectively collect the body fluids and thereby preventing the leakage of the body fluids onto the undergarment of the user. The present disposable sanitary pad 100 can be utilized to conveniently and efficiently solve the problems faced by the wearers, which include the proper placement of the disposable sanitary pad 100 within the undergarment of the user or the wearer, prolonged usage of the disposable sanitary pad 100, etc. The present disposable sanitary pad 100 and the method of using the disposable sanitary pad 100 helps the users to wear the sanitary pad 100 for long periods of time, away from the user's skin, but providing adequate absorption of the body fluids, prevents leakage of the body fluids to the undergarments of the user and allows easy and hygienic disposal of the disposable sanitary pad 100 after use. The present disposable sanitary pad 100 further helps the user to comfortably place the pad 100, stretched within the undergarment, thereby preventing the shifting or dislocation or movement of the pad 100 when worn by the user.

FIG. 1 further illustrates the different layers and parts of the disposable sanitary pad 100 that the users can comfortably place within the undergarment of the user, according to a preferred embodiment of the present invention. The present disposable sanitary pad 100 configured to removably attach with the undergarment of the user includes a central pad portion 102 having a substantially oblong shape to place under the crotch area of the user or the wearer. The disposable sanitary pad 100 including central pad portion 102 is made available in different sizes such as small, medium, large and extra large sizes for use by different age groups and to fit with all kinds of body shapes of the users. The size and shape of the disposable sanitary pad 100 provides proper covering of the crotch area of the user to collect and absorb the body fluids produced by the user. However, in all different sizes, the shape, different parts including the fluid absorption layers for absorbing the body fluids used in the disposable sanitary pad 100 remain the same. The central pad portion 102 of the disposable sanitary pad 100 includes three major layers, such as a fluid impervious flexible base 104, a number of absorbent layers 106 and a fluid pervious layer 108, which is at the top or top surface of the central pad portion 102. The fluid pervious layer 108 covers the underlying absorbent layers 106 and passes the body fluids downwards for absorption by the absorbent layers 106. In a preferred embodiment, the disposable sanitary pad 100 including the central pad portion 102 having the fluid impervious flexible base 104, the absorbent layers 106 and the fluid pervious layer 108 attached to the fluid impervious flexible base 104 are made from good quality, rash free, allergy free, hygienic materials. In a preferred embodiment, the fluid impervious flexible base 104 is provided with a substantially oblong shape sufficient to longitudinally cover the crotch area of the user. The fluid impervious flexible base 104 has an interior surface and an exterior surface and supports the top layers including the absorbent layers 106 for absorbing the body fluids and the fluid pervious layer 108 covering the absorbent layers 106. In a preferred embodiment, the absorbent layers 106 are made from one or more absorbent materials including cellulose. In addition, the fluid pervious layer 108 is made from soft and comfortable, rash free, allergy free and hygienic materials to enable the user to comfortably wear the disposable sanitary pad for longer duration. In a preferred embodiment, the surface area or the size of the fluid impervious flexible base 104 is more than that of the absorbent layers 106 and the fluid pervious layer 108 when stacked together. This ensures that the fluid impervious flexible base 104 has an extended portion around the edges or the periphery of both the absorbent layers 106 and the fluid pervious layer 108, which provides adequate coverage for the crotch area of the undergarment and the user.

In a preferred embodiment of the present disclosure, the absorbent layers 106 of the central pad portion 102 are placed on the interior surface of the fluid impervious flexible base 104. The fluid pervious layer 108, which includes pores to pass the body fluids of the user to the absorbent layers 106 are placed over the absorbent layers 106. The edges of the fluid pervious layer 108 and the absorbent layers 106 are attached together using an attachment means, such as but not limited to, stitching, gluing and other forms of attachment means. This ensures that the absorbent layers 106 capable of absorbing the body fluids of the user are properly sandwiched between the fluid impervious flexible base 104 and the fluid pervious layer 108 to give comfort to the wearer at the same time provide adequate protection from the body fluids. In some instances, the fluid impervious flexible base 104 has a layer of fluid absorbing materials, which further improves the effectiveness of the present disposable sanitary pad 100 by absorbing the body fluids, in case if the body fluids leak outside the absorbent layers 106 of the central pad portion 102. In some other instances, only the interior surface of the absorbent layers 106 are made from fluid absorbing materials and are properly designed at the edges of the absorbent layers 106 to prevent leakage of the body fluids into the underlying fluid impervious flexible base 104.

The fluid impervious flexible base 104 is provided with an oblong shape having a top portion, a bottom portion and a pair of side portions to properly cover the crotch area of the undergarment of the user. The top portion and bottom portion at the exterior surface of the fluid impervious flexible base 104 is provided with adhesive strips 110a, 110b, respectively, to enable the users to properly attach the disposable sanitary pad 100 to the crotch area of the undergarment of the user. The adhesive strips 110a at the top portion and the adhesive strip 110b at the bottom portion of the exterior surface of the fluid impervious flexible base 104 attaches the disposable sanitary pad 100 in a longitudinal stretched fashion covering the crotch area of the undergarment of the user and maintains the position even after long periods of use, during wet conditions and during physical activities performed by the user. The fluid impervious flexible base 104 of the disposable sanitary pad 100 further includes a pair of flexible wings 112 attached to the pair of side portions of the fluid impervious flexible base 104. In a preferred embodiment of the present invention, the flexible wings are attached or stitched to the interior surface of the fluid impervious flexible base 104 and extends towards and beyond the pair of adjacent edges on either side of the fluid impervious flexible base 104. The flexible wings 112 or a portion of the wings 112 or multiple locations on the flexible wings 112 are covered with an adhesive to attach with the undergarment of the user to maintain the stretched position of the disposable sanitary pad 100, laterally covering the crotch area of the undergarment. The exterior surface of the fluid impervious flexible base 104 of the disposable sanitary pad 100 is further provided with a pair of elongated adhesive strips 114. The pair of elongated adhesive strips 114 is longitudinally attached to the exterior surface of the fluid impervious flexible base 104 on either sides of a center portion of the disposable sanitary pad 100 to hold the central pad portion 102 at the center portion of the crotch area of the undergarment. Further in a preferred embodiment, a number of corners 118a, 118b, 118c and 118d at the edges of the plurality of absorbent layers 106 and the fluid pervious layer 108 attached to the fluid impervious flexible base 104 is coated with an adhesive material for removably attaching the disposable sanitary pad onto the undergarment of the user. The adhesive material provided at the corners 118a, 118b, 118c and 118d at the edges of the absorbent layers 106 and the fluid pervious layer 108 is at the exterior surface of the fluid impervious flexible base 104 so that the adhesives get directly in contact with the interior of the undergarment. The adhesive strips 110a, 110b attached to the top portion and the bottom portion and the adhesive material provided at the corners 118a, 118b, 118c and 118d of the absorbent layers 106 and the fluid pervious layer 108 at the exterior surface of the fluid impervious flexible base 104 removably attaches the central pad portion 102 in a longitudinal orientation covering the crotch area of the undergarment of the user. This attachment ensures that the disposable sanitary pad stays in place within the undergarment of the user, covering the crotch area, for prolonged periods and at different conditions including wet and during movement of the users. The present disposable sanitary pad 100 is further provided with a pouch 116 removably attached to the exterior surface of the fluid impervious flexible base 104 to store one or more deodorized wet wipes for use by the user during handling and disposal of the disposable sanitary pad 100. The users can detach the pouch 116 by pulling it out of the exterior surface of the fluid impervious flexible base 104 before using the disposable sanitary pad 100 with the undergarment of the user.

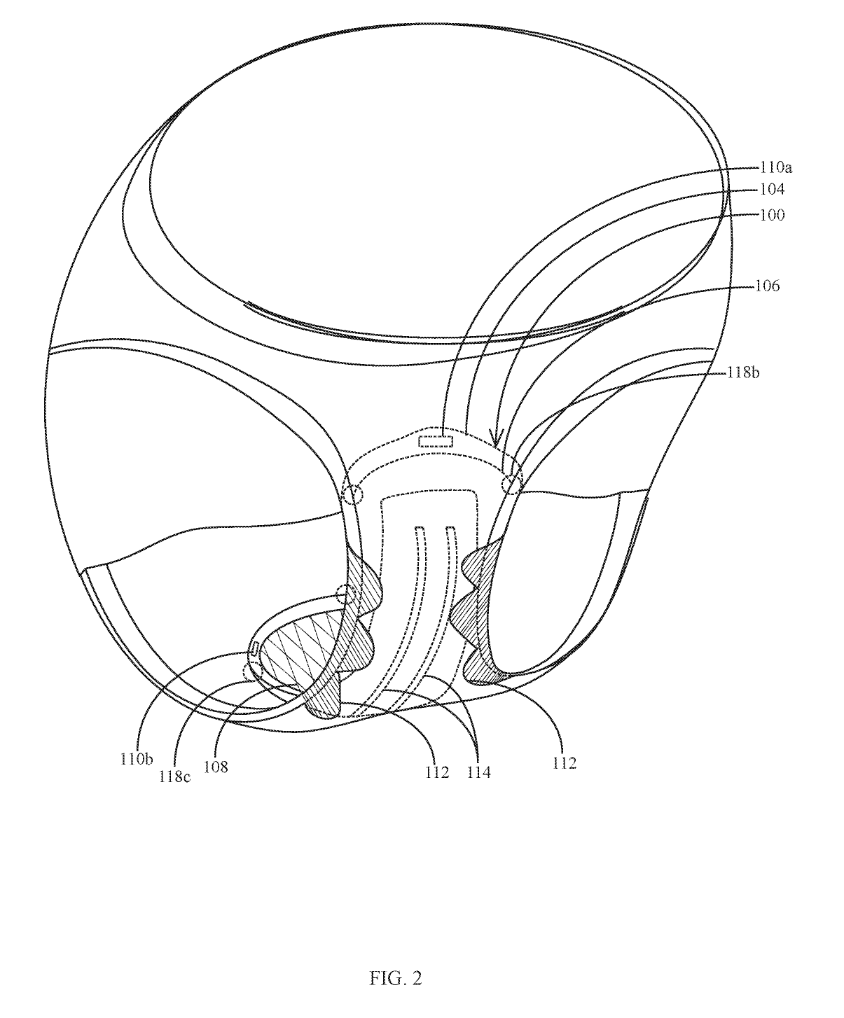

FIG. 2 illustrates the disposable sanitary pad 100 attached within the undergarment of the user, according to a preferred embodiment of the present invention. In an embodiment of the present disposable sanitary pad 100, the outer edge portions of the fluid impervious flexible base 104 extends outwards around the edges of the absorbent layers 106 and the fluid pervious layer 108 attached to the fluid impervious flexible base 104. This way the outer edge portions of the fluid impervious flexible base 104 around the edges of the absorbent layers 106 and the fluid pervious layer 108 forms an extra barrier or protective area to prevent the leakage of the body fluid to the undergarments of the user. In some embodiment, the outer edges of the absorbent layers 106 and the fluid pervious layer 108 are provided with raised contour shapes to channel the body fluids and to allow the body fluids to pass directly through the fluid pervious layer 108 into the absorbent layers 106 attached to the fluid impervious flexible base 104 using the attachment means including stitching and adhesives. These contours provide additional comfort to the wearer and prevent leakage or spillage of the body fluids over the edges to the undergarments of the wearer or the user. Further the corners 118a, 118b, 118c and 118d at the edges of the absorbent layers 106 and the fluid pervious layer 108 attached together to the fluid impervious flexible base 104 is coated with adhesive material at the exterior surface of the fluid impervious flexible base 104 for removably attaching the disposable sanitary pad 100 into the undergarment of the user.

The top portion and the bottom portion of the exterior surface of the fluid impervious flexible base 104 are also provided with the adhesive strips 110a, 110b as shown in FIG. 1.

The top portion of the exterior surface of the fluid impervious flexible base 104 comprises an area extending from the attached edges of the absorbent layers 106 and the fluid pervious layer 108 towards an adjacent top edge portion of the fluid impervious flexible base 104. Similarly the bottom portion of the exterior surface of the fluid impervious flexible base 104 comprises an area extending from the attached edges of the absorbent layers 106 and the fluid pervious layer 108 towards an adjacent bottom edge portion of the fluid impervious flexible base 104. The pair of side portions of the exterior surface of the fluid impervious flexible base 104 includes an area extending from the attached edges of the absorbent layers 106 and the fluid pervious layer 108 towards an adjacent left and right edge portions of the fluid impervious flexible base 104 as shown in FIG. 2. The pair of elongated adhesive strips 114, the adhesives provided at the corners 118a, 118b, 118c and 118d and the adhesive strips 110a, 110b attached to the top and bottom portion of the exterior surface of the fluid impervious flexible base 104 removably attaches the central pad portion 102 in a longitudinal orientation to cover the crotch area of the undergarment of the user. The adhesives form a stable attachment with the interior of the undergarment of the user and help to maintain the longitudinal orientation of the disposable sanitary pad 100 within the undergarment and cover the crotch area of the undergarment and the user. The flexible wings 112 on the pair of side portions of the fluid impervious flexible base 104 is designed to fold over and stick to the undergarment portion on either side of the crotch area of the undergarment. The flexible wings 112 are covered using a pair of removable papers 120 to prevent undesired sticking of the flexible wings 112 to other parts of the disposable sanitary pad 100 and the clothing, skin and the undergarment of the user. The adhesives provided on the flexible wings 112 further enable the attachment of the flexible wings 112 to the pair of side portions of the fluid impervious flexible base 104 and to the side edges and underside portion on either side of the crotch area of the undergarment worn by the user. The adhesives provided on one or more locations on the flexible wings 112 enables the user to fold over and attach the flexible wings 112 to the side edges and underside portion of the undergarment of the user. This way the flexible wings 112 attached to the pair of side portions of the fluid impervious flexible base 104 and to the undergarment portion on either sides of the crotch area of the undergarment worn by the user laterally or transversally stretches the disposable sanitary pad 100 thereby effectively preventing leakage of the body fluids on either side of the crotch area of the undergarment and to the legs or clothing of the user. The present disposable sanitary pad 100 cover the crotch area of the undergarment of the user and the flexible wings 112 extending towards the pair of legs of the user, which also helps to channel the body fluids towards the absorbent layers 106 of the central pad portion 102.

The pouch 116 carrying the deodorized wet wipes can be detached before using the disposable sanitary pad 100. In some instances, the deodorized wet wipes are also coated with sanitizers or disinfectants for killing any microbes present in the hands or the pad while handling the disposable sanitary pad 100 for disposal. Further the disposable sanitary pad 100 is made available in a variety of sizes including small, medium, large and extra large sizes for use by users having different types of body shapes and sizes. In each of the sizes, the pouch 116 associated with the disposable sanitary pad 100 is appropriately sized with appropriately sized deodorized wet wipes. The sizes of the deodorized wet wipes associated with each of the disposable sanitary pad 100 varies from small, medium, large and extra large sizes, which would allow the users to cover the disposable sanitary pad 100 after use and dispose the disposable sanitary pad 100 with hygiene. The users can cover the disposable sanitary pad 100 using the deodorized wet wipes and detach the disposable sanitary pad 100 from the undergarment after use and can be easily disposed. The deodorized wet wipes also gives a pleasant smell to the hands of the user. In some instances, the deodorized wet wipes provided within the pouch 116 of the disposable sanitary pad 100 are available in different flavors including lavender, jasmine etc. In some other instances, the pouch 116 can further be utilized to collect and dispose the disposable sanitary pad 100 after use. In some other instances, the pouch 116 is sufficiently sized to collect the disposable sanitary pad 100 after use for disposal and is made from biodegradable materials.

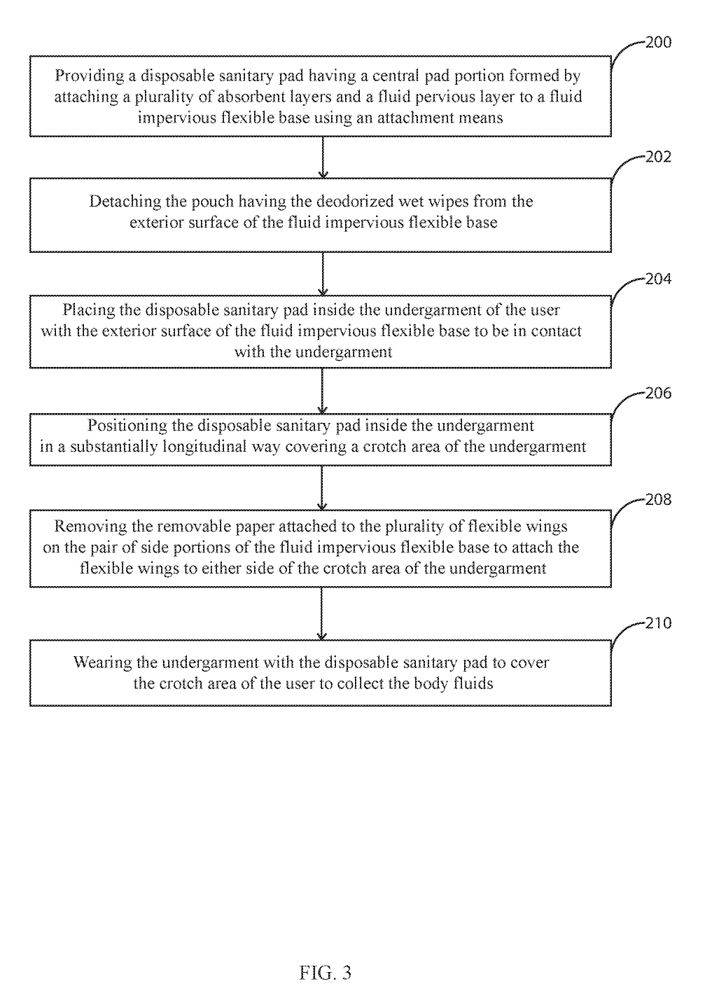

FIG. 3 illustrates a method of comfortably placing the disposable sanitary pad 100 within the undergarment of the user, according to a preferred embodiment of the present invention. The method includes the steps of providing the disposable sanitary pad 100 as shown in block 200. The disposable sanitary pad 100 includes the central pad portion 102 formed by attaching the number of absorbent layers 106 and the fluid pervious layer 108 to a fluid impervious flexible base 104 using the attachment means such as stitching. However, other forms of attachment methods, such as, but not limited to, gluing, can also be utilized to properly position the absorbent layers 106 on the interior surface of the fluid impervious flexible base 104. Further the fluid pervious layer 108, which forms the outer layer of the central pad portion 102 and in contact with the crotch area of the user or the wearer covers the absorbent layers 106 and attaches to the fluid impervious flexible base 104 of the central pad portion 102. The fluid pervious layer 108 is made from a porous material or clothing that would easily allow the flow of the body fluid to the underlying absorbent layers 106. In a preferred embodiment of the present disposable sanitary pad 100, the outer edge portions of the fluid impervious flexible base 104 extends out to surround the edges of the absorbent layers 106 and the fluid pervious layer 108 attached to the fluid impervious flexible base 104.

The disposable sanitary pad 100 is attached with the pair of adhesive strips 110a, 110b at the top and the bottom portion of the exterior surface of the fluid impervious flexible base 104. The disposable sanitary pad 100 further includes the pair of elongated adhesive strips 114 longitudinally attached to the exterior surface of the fluid impervious flexible base 104 and adhesives at the corners 118a, 118b, 118c and 118d of the absorbent layers 106 and a fluid pervious layer 108 attached to the fluid impervious flexible base 104. The fluid impervious flexible base 104 is attached with the flexible wings 112 at the pair of side portions and is coated with an adhesive on the surface and covered using a removable paper as in FIG. 2. The disposable sanitary pad 100 is provided with the pouch 116 for storing the deodorized wet wipes. The pouch 116 is removably attached to the exterior surface of the fluid impervious flexible base 104. The users can detach the pouch 116 from the exterior surface of the fluid impervious flexible base 108 before wearing the undergarment, as shown in block 202. After detaching the pouch 116, the users can place the disposable sanitary pad 100 inside the undergarment of the user, with the exterior surface of the fluid impervious flexible base 104 of the disposable sanitary pad 100 in contact with the undergarment, which is shown in block 204. The users can then position the disposable sanitary pad 100 inside the undergarment in a substantially longitudinal way covering a crotch area of the undergarment of the user, as in block 206. The disposable sanitary pad 100 is positioned at the center of the crotch area of the undergarment by attaching the pair of adhesive strips 110a, 110b at the top and bottom portion of the exterior surface of the fluid impervious flexible base 104 and the pair of elongated adhesive strips 114 to the undergarment of the user, which is shown in block 208. In the above step, the adhesives at the corners 118a-118d of the absorbent layers 106 and the fluid pervious layer 108 gets attached to the undergarment of the user. After properly placing the disposable sanitary pad 100 within the undergarment, covering the crotch area of the undergarment, the users can peel the removable paper 120 attached to the flexible wings 112 on the pair of side portions of the fluid impervious flexible base 104 to attach the wings 112 to either side of the crotch area of the undergarment, which is shown in block 210. This prevents the movement of the disposable sanitary pad 100 in the left and right directions away from the crotch area of the undergarment of the user. After the above steps, the users can wear the undergarment with the disposable sanitary pad 100 to cover the crotch area and the flexible wings 112 extending towards the pair of legs of the user to effectively collect the body fluids from the user, which is shown in block 212. The flexible wings 112, the pair of adhesive strips 110a, 110b and the pair of elongated adhesive strips 114 attached to the undergarment of the user maintains the position of the disposable sanitary pad 100 during movement and sitting postures of the user to prevent the leakage of the body fluids to the undergarment of the user. The disposable sanitary pad 100 can be easily detached from the undergarment after use and disposed by covering the disposable sanitary pad 100 using the deodorized wet wipes retrieved from the pouch 116.

It is noted that the lobed tabs 112 enable a greater flexibility by providing for separation between the different lobs 112 thus allowing the garment material to stretch therebetween.

Further, it should be noted that the steps described in the method of use could be carried out in many different orders according to user preference. The use of “step of” should not be interpreted as “step for”, in the claims herein and is not intended to invoke the provisions of 35 U.S.C. § 112, (6). Upon reading this specification, it should be appreciated that, under appropriate circumstances, considering such issues as design preference, user preferences, marketing preferences, cost, structural requirements, available materials, technological advances, etc., other methods of use arrangements such as, for example, different materials and designs within above-mentioned list, elimination or addition of certain steps, including or excluding certain maintenance steps, etc., may be sufficient.

The foregoing description of the preferred embodiment of the present invention has been presented for the purpose of illustration and description. It is not intended to be exhaustive or to limit the invention to the precise form disclosed. Many modifications and variations are possible in light of the above teachings. It is intended that the scope of the present invention not be limited by this detailed description, but by the claims and the equivalents to the claims appended hereto.

What is claimed is:

1. A disposable sanitary pad configured to removably attach with an undergarment of a user comprising:
a) a central pad portion having:
i. a fluid impervious flexible base with a substantially oblong shape having an interior surface and an exterior surface;
ii. a plurality of absorbent layers placed on the interior surface of the fluid impervious flexible base; and
iii. a fluid pervious layer covering the top of the plurality of absorbent layers to allow passage of at least one body fluid of the user to the underlying absorbent layers,
wherein a plurality of edges of the plurality of absorbent layers and the fluid pervious layer are attached to the fluid impervious flexible base using an attachment means;
b) a pair of adhesive strips attached to a top portion and a bottom portion of the exterior surface of the fluid impervious flexible base;
c) a plurality of flexible wings attached to a pair of side portions of the fluid impervious flexible base,
wherein the plurality of wings is coated with an adhesive at a plurality of locations to attach with the undergarment of the user;
d) a pair of elongated adhesive strips longitudinally attached to the exterior surface of the fluid impervious flexible base to hold the central pad portion at a center portion of a crotch area of the undergarment; and
e) at least one pouch removably attached to the exterior surface of the fluid impervious flexible base to store a plurality of deodorized wet wipes for use during handling and disposal of the disposable sanitary pad after use;
wherein the disposable sanitary pad is longitudinally placed inside the undergarment using the pair of adhesive strips, the pair of elongated adhesive strips and the plurality of flexible wings to protect the crotch area of the undergarment and area on either sides of the crotch area towards the pair of legs of the user.

2. The disposable sanitary pad of claim 1 wherein a plurality of outer edge portions of the fluid impervious flexible base extends outwards around the edges of the plurality of absorbent layers and the fluid pervious layer attached to the fluid impervious flexible base.

3. The disposable sanitary pad of claim 1 wherein the plurality of edges of the absorbent layers and the fluid pervious layer are attached to the fluid impervious flexible base using the attachment means including stitching and adhesives.

4. The disposable sanitary pad of claim 1 wherein the plurality of absorbent layers and the fluid pervious layer are provided with a plurality of contours to completely absorb the body fluids when in contact with the body of the user.

5. The disposable sanitary pad of claim 1 wherein the plurality of absorbent layers, the fluid pervious layer and the fluid impervious flexible base are made from rash free, allergy free and odor free materials for use in wet and dry conditions.

6. The disposable sanitary pad of claim 1 wherein a plurality of corners at the edges of the plurality of absorbent layers and the fluid pervious layer attached to the fluid impervious flexible base is coated with an adhesive material for removably attaching onto the undergarment of the user,
wherein the adhesive material provided at the plurality of corners of the absorbent layers and the fluid pervious layer is at the exterior surface of the fluid impervious flexible base.

7. The disposable sanitary pad of claim 6 wherein the adhesive strips attached to the top portion and the bottom portion and the adhesive material provided at the plurality of corners of the absorbent layers and the fluid pervious layer at the exterior surface of the fluid impervious flexible base removably attaches the central pad portion in a longitudinal orientation covering the crotch area of the undergarment of the user.

8. The disposable sanitary pad of claim 1 wherein the pair of elongated adhesive strips longitudinally attached to the exterior surface of the fluid impervious flexible base hold the central pad portion at the center portion of the crotch area of the undergarment during different postures and during a plurality of activities of the user.

9. The disposable sanitary pad of claim 1 wherein the adhesives provided at the plurality of locations on the plurality of wings enables the user to attach the wings to the undergarment of the user.

10. The disposable sanitary pad of claim 1 wherein the wings on the pair of side portions of the fluid impervious flexible base is designed to fold over and stick to the undergarment portion on either side of the crotch area of the user.

11. The disposable sanitary pad of claim 1 wherein the wings are covered using a pair of removable papers to enable the attachment of the wings to the pair of side portions of the fluid impervious flexible base and to the undergarment portion on either sides of the crotch area of the user.

12. The disposable sanitary pad of claim 1 wherein the wings attached to the pair of side portions of the fluid impervious flexible base and to the undergarment portion on either sides of the crotch area of the undergarment stretches the central pad portion in a lateral direction to prevent leakage of the body fluids on either sides of the crotch area of the undergarment.

13. The disposable sanitary pad of claim 1 wherein the pouch carrying the plurality of deodorized wet wipes can be detached before using the disposable sanitary pad.

14. The disposable sanitary pad of claim 1 is available in a plurality of sizes including small, medium, large and extra large sizes,
wherein the deodorized wet wipes and the carrying pouch associated with each of the disposable sanitary pad varies in sizes including small, medium, large and extra large sizes.

15. The disposable sanitary pad of claim 1 wherein the pouch is further utilized to store and dispose the disposable sanitary pad after use.

16. The disposable sanitary pad of claim 1 wherein the plurality of absorbent layers are made from a plurality of absorbent materials including cellulose.

17. A method of using a disposable sanitary pad with an undergarment of a user comprising the steps of:
a. providing the disposable sanitary pad having:
i. a central pad portion formed by attaching a plurality of absorbent layers and a fluid pervious layer to a fluid impervious flexible base using an attachment means,
wherein a plurality of outer edge portions of the fluid impervious flexible base extends out to surround a plurality of edges of the absorbent layers and the fluid pervious layer attached to the fluid impervious flexible base,
wherein an exterior surface of the fluid impervious flexible base is provided with adhesives at a plurality of corners of the edges of the absorbent layers and the fluid pervious layer;
ii. a pair of adhesive strips attached to a top portion and a bottom portion of an exterior surface of the fluid impervious flexible base;
iii. a plurality of flexible wings attached to a pair of side portions of the fluid impervious flexible base,
wherein the plurality of wings is coated with an adhesive on at least one surface and covered using a removable paper;
iv. a pair of elongated adhesive strips longitudinally attached to a center portion of the exterior surface of the fluid impervious flexible base; and
v. at least one pouch storing a plurality of deodorized wet wipes removably attached to the exterior surface of the fluid impervious flexible base;
b. detaching the pouch having the deodorized wet wipes from the exterior surface of the fluid impervious flexible base;
c. placing the disposable sanitary pad inside the undergarment of the user with the exterior surface of the fluid impervious flexible base to be in contact with the undergarment;
d. positioning the disposable sanitary pad inside the undergarment in a substantially longitudinal way covering a crotch area of the undergarment,
wherein the disposable sanitary pad is positioned at the center of the crotch area of the undergarment by attaching the pair of adhesive strips at the top and bottom portion, the pair of elongated adhesive strips and the plurality of corners of the absorbent layers and the fluid pervious layer at the fluid impervious flexible base to the undergarment of the user;
e. peeling the removable paper attached to the plurality of flexible wings on the pair of side portions of the fluid impervious flexible base to attach the flexible wings to either side of the crotch area of the undergarment; and
f. wearing the undergarment with the disposable sanitary pad to cover the crotch area of the user to collect the body fluids.

18. The method of claim 17 wherein the flexible wings, the pair of adhesive strips, the pair of elongated adhesive strips and the adhesives at the plurality of corners of the absorbent layers and the fluid pervious layer attached to the undergarment of the user maintains the position and shape of the disposable sanitary pad to prevent the leakage of the body fluids to the undergarment of the user.

19. The method of claim 17 wherein the disposable sanitary pad is detached from the undergarment and disposed after use with hygiene by covering the disposable sanitary pad using the deodorized wet wipes from the pouch.
